(19) United States
(12) Patent Application Publication
(10) Pub. No.: US 2003/0232394 A1
(43) Pub. Date: Dec. 18, 2003

(54) High throughput screening method and assay system for determining the interaction between C-reactive protein and components binding to it

(75) Inventors: Aimo Kannt, Frankfurt (DE); Antje Pommereau, Otterberg (DE)

Correspondence Address: ROSS J. OEHLER, AVENTIS PHARMACEUTICALS INC., ROUTE 202-206, MAIL CODE: D303A, BRIDGEWATER, NJ, 08807, US

(73) Assignee: Aventis Pharma Deutschland GMBH, Frankfurt am Main (DE)

(21) Appl. No.: 10/459,245

(22) Filed: Jun. 11, 2003

Related U.S. Application Data
(60) Provisional application No. 60/426,945, filed on Nov. 15, 2002.

(30) Foreign Application Priority Data
Jun. 12, 2002 (DE) 10226011.7

(51) Int. Cl. G01N 33/53

(52) U.S. Cl. 435/7.1

(57) ABSTRACT

Disclosed are methods and assay systems for high throughout screening of interactions between C-reactive protein, C1q, and components binding to them.

This application claims the benefit of priority under 35 U.S.C. §119 of German Application No. 10226011.7 filed on Jun. 12, 2002, and of U.S. Provisional Application No. 60/426,945 filed on Nov. 15, 2002, the contents of both of which are hereby incorporated by reference.

### FIELD OF THE INVENTION

Embodiments of the present invention relate to high-throughput-screening (HTS) capable methods and assay systems for determining the interaction between C-reactive protein (CRP) and C1q and components binding to CRP and C1q, respectively. The methods and assay systems are useful for, for example, determining the concentration of a solution containing CRP and C1q, respectively, and for determining substances which influence the interaction of CRP and C1q, respectively, and of the components binding thereto.

### BACKGROUND OF THE INVENTION

CRP is an acute-phase plasma protein whose serum concentration increases rapidly and greatly after an infection or tissue injury (Volanakis (2001), Molecular Immunology 38, 189-197). CRP binds to phosphocholine (PCh) in the presence of Ca²⁺ ions. Such ions are very common in polysaccharides of pathogenic organisms and in cell membranes of damaged and necrotic cells. CRP bound to PCh can activate the classical complement cascade by binding to the protein C1q.

The complement is part of the immune system and primarily involved in the antibody-mediated immune defenses. The three physiological functions of the complement are the defense against bacterial infections, the connection of congenital and acquired immunity and the removal of immunocomplexes and apoptotic cells. A distinction is made between the classical, the alternative and the mannose-lectin complement cascades (Walport (2001), N Engl J Med 344, 1058-1066). The classical complement cascade leads to the lysis of bacterial cells and starts with C1q associating with an antibody binding to the cell surface or with PCh-bound CRP.

C-reactive protein is used as a marker and, in addition, as a predictor for coronary heart disease, the most common cause of mortality in industrialized countries (Aifai and Aidker (2001), Clinical Chemistry 47,403-411).

New studies (Jialal et al (2001), Circulation 103, 1933-1935) suggest that lowering the CRP level or blocking the CRP-mediated effector functions, for example complement activation due to binding to C1q, may be useful for the prevention and treatment of coronary heart disease.

It is therefore desirable firstly to provide a method which allows determining the concentration of CRP and/or C1q in blood plasma in a simple manner and, secondly, allows identification of substances which act on the interaction of CRP and/or C1q with other components, in particular on the interaction between CRP and C1q, in a modulating, i.e. activating or inhibiting, way.

Disclosed herein is a method which makes possible a high throughput screening for substances which influence complement activation due to binding of CRP to C1q.

### BRIEF DESCRIPTION OF THE FIGURES

FIG. 1 illustrates an assay for determining the binding of CRP to C1q according to one embodiment of the invention. A donor component is located on particles bound to anti-C1q antibody which in turn is bound to C1q. An acceptor component is located on particles bound to goat anti-rabbit antibody; the goat anti-rabbit antibody is bound to rabbit anti-CRP antibody which in turn is bound to CRP. Light that impinges on the acceptor component causes it to convert ambient oxygen to singlet oxygen. Binding between CRP and C1q brings the donor and acceptor components sufficiently close together to allow the singlet oxygen to reach the acceptor component, causing the component to release light. The light is detectable with standard photodetection equipment, such as a photomultiplier.

### DETAILED DESCRIPTION OF THE INVENTION

According to one embodiment of the invention, one determines a binding event of two components binding directly or via one or more components to CRP or C1q by performing the following steps:

(a) initially introducing a CRP- or C1q-containing solution;

(b) adding at least one donor component or a group of components containing at least one donor component to the CRP- or C1q-containing solution, the donor component comprising at least one compound or group of compounds capable of emitting a signal after excitation by a light source (donor group) and the donor component being capable of binding directly or via one or more components from the group of components to CRP or C1q;

(c) adding at least one acceptor component or a group of components containing at least one acceptor component to the CRP- or C1q-containing solution, the acceptor component comprising at least one compound or group of compounds capable of receiving and emitting in the form of electromagnetic radiation the signal emitted by the compound or group of compounds according to (b) (acceptor group) and the acceptor component being capable of binding directly or via one or more components of the group of components to CRP or C1q;

(d) exciting the at least one compound or group of compounds according to (b) (donor group) by means of a light source;

(e) detecting the electromagnetic radiation emitted by the at least one compound or group of compounds according to (c) (acceptor group) in order to detect the binding event.

The emitted electromagnetic radiation according to (c) is here preferably fluorescence radiation. The light source according to (d) may be, for example, a laser or a lamp, for example a helium or halogen lamp. The electromagnetic radiation according to (e) may be detected, for example, with the aid of a photomultiplier.

The components to be used according to the invention preferably are or comprise polypeptides. In a particularly preferred embodiment of the invention, at least one of the components is an antibody which may be a monoclonal or polyclonal antibody. If a group of components is used, the components are preferably capable of binding to CRP or C1q or to one another in spatial succession. The binding takes place in the form of a binding cascade, an example of which is the successive binding of antibodies (primary antibody, secondary antibody, etc.). The components binding directly to CRP or C1q according to (b) and (c) are here preferably components which bind in each case to a particular binding region or to a particular epitope of the CRP or C1q, the CRP- or C1q-binding component according to (b) binding to a different binding site than the CRP- or C1q-binding component according to (c).

In another preferred embodiment of the method of the invention, one of the components binding directly to CRP or C1q is a naturally occurring binding partner of the CRP or C1q, which, in case of the CRP is preferably C1q and vice versa. If the donor or acceptor component is bound directly to CRP or C1q, then, in this case, either the donor or the acceptor component is accordingly either C1q or CRP comprising a donor or acceptor group. When using a group of components capable of forming a binding cascade, C1q or CRP is accordingly the first component binding to CRP or C1q via which it is possible to bind either the donor or the acceptor component to CRP or C1q. The remaining components of the group of components are preferably antibody molecules.

Thus, for example, when initially introducing a CRP-containing solution, preferably either the donor or the acceptor component itself comprises C1q or the donor or acceptor component is bound via C1q to CRP. In this case, the donor or acceptor component may comprise an anti-C1q antibody or a secondary antibody capable of binding to the anti-C1q antibody. Accordingly, the in each case other component (acceptor or donor component) then comprises an anti-CRP antibody or an antibody capable of binding to the anti-CRP antibody or a tertiary antibody directed against the latter.

In a preferred embodiment, the at least one compound or group of compounds according to (b) (donor group) and/or according to (c) (acceptor group) are localized on particles, with the average diameter of the particles preferably being between 150 and 250 nm, preferably at approximately 200 nm. Accordingly, the donor or acceptor components then comprise the particles which, in this case, may be made of a polymeric material.

The method of the invention is an HTS (high throughput screening)-capable method which may be carried out homogeneously in the form of a one-step method. In a preferred embodiment, the method of the invention is carried out according to the “mix and measure” principle. A particular characteristic feature of the novel method is the possibility of monitoring binding between the two proteins in solution, since none of the reactants needs to be immobilized on a solid phase. Moreover, it is possible to measure the proteins obtained from biological sources directly, without the need for modifying CRP or C1q, for example by binding of a fluorophor. The protein can therefore (although need not) be subjected in its native state to the assay, thus removing the risk of denaturation which would be present in the case of immobilizing and/or modifying the protein.

One may employ a reduced sample volume of the method of the invention compared to conventional methods. Thus it is possible to use volumes of less than 10 μl. Moreover, it is possible to use highly diluted solutions with CRP or C1q concentrations of less than 1 nM, and even less than 100 pM.

In a preferred embodiment of the method of the invention, the signal is transferred from the at least one compound or group of compounds according to (b) (donor group) to the at least one compound or group of compounds according to (c) (acceptor group) via singlet oxygen. In this case, the donor group preferably comprises a compound which can convert triplet oxygen to singlet oxygen after excitation by a laser, and the acceptor group comprises at least one first compound excitable by singlet oxygen and a second compound capable of absorbing in an emissionless manner and emitting in the form of fluorescence radiation the energy absorbed by the group excitable by singlet oxygen.

The singlet oxygen formed may diffuse from the donor group to the acceptor group and react with the chemiluminescent substances present there. The energy released in the process is transferred to the fluorophores which finally emit the energy in the form of fluorescence radiation which can be detected using a photomultiplier. A precondition for a detectable signal is the spatial proximity of donor and acceptor beads, since singlet oxygen is unstable and decays in aqueous solution. Therefore, the binding components to be used in this method are chosen in such a way that, when the binding event occurs, the distance between the donor and acceptor groups is preferably less than 200 nm.

Particular preference is given in this embodiment to donor group and acceptor group localized on particles, the particles preferably having an average diameter of between 150 and 250 nm, preferably of approximately 200 nm. The preferred use of the particles here is such that the final concentration of donor group-carrying particles is 1-40 μg/ml and the final concentration of acceptor group-carrying particles is 1-80 μg/ml. The binding capacity of the particles may be, for example, from approximately 0.1 nM to 1 nM per μg/ml particles.

Particular preference is given to using AlphaScreen beads from Perkin-Elmer Life Sciences as particles in the method. In this embodiment, the donor group is excited by irradiating at a wavelength of 680 nm, using a laser, and the radiation emitted by the acceptor group can be detected at a wavelength between 520 and 600 nm. Accordingly, it is possible, in this case, for the method to be carried out in such a way that donor and acceptor beads carrying donor and acceptor groups are introduced initially. Components capable of binding either themselves or via further components to CRP or C1q are then bound to the donor and acceptor beads. As already mentioned above, these components may be either C1q or CRP or may be an anti-CRP or anti-C1q antibody or else may be a secondary antibody directed against the anti-CRP or anti-C1q antibody.

In a further preferred embodiment of the method of the invention, the signal is transferred from the at least one compound or group of compounds according to (b) (donor group) to the at least one compound or group of compounds according to (b) (acceptor group) via emissionless energy transfer, preferably via fluorescence resonance energy transfer. In this case it is possible to carry out the method, for example, in such a way that the at least one compound or group of compounds according to (b) (donor group) comprises a europium salt-containing compound and that the at least one compound or group of compounds according to (c) (acceptor group) may comprise allophycocyanine (Grepin et al. (2000), Drug Discovery Today 5,212).

Alternatively, the donor and acceptor groups may be dyes suitable for emissionless energy transfer. It is furthermore possible to use all compounds known to the skilled worker which are suitable for emissionless energy transfer, in particular for fluorescence resonance energy transfer (see, for example, Pope et al. (1999), Drug Discovery Today 4,350). In this embodiment, preference is given to choosing the binding components to be used in such a way that the distance between the donor and acceptor groups after binding is less than 10 nm, since an effective emissionless energy transfer is not possible at a larger distance. In an embodiment preferred according to the invention, the method of the invention is used for determining the concentration of a CRP- or C1q-containing solution with unknown CRP or C1q content, the solution being preferably blood plasma or blood serum or being blood plasma or blood serum diluted by means of a suitable physiological buffer or by means of water. The method may be used, for example, for diagnostic purposes, in particular for determining the state of inflammation of an organism and/or for determining the risk of cardiac arrest and/or stroke.

The present invention therefore likewise relates to an HTS-capable, diagnostic assay system for determining a binding event between CRP or C1q and a component binding to CRP or C1q, comprising the following components:

(a) at least one donor component or group of components containing at least one donor component, the donor component comprising at least one compound or group of compounds capable of emitting a signal after excitation by a light source (donor group) and the donor component being capable of binding directly or via one or more components from the group of components to CRP or C1q; and

(b) at least one acceptor component or group of components containing at least one acceptor component, the acceptor component comprising at least one compound or group of compounds capable of receiving and emitting in the form of electromagnetic radiation the signal emitted by the compound or group of compounds according to (b) and the acceptor component being capable of binding directly or via one or more components of the group of components to CRP or C1q.

The HTS-capable assay system here preferably comprises the following additional components:

(c) blood serum or blood plasma, or blood diluted in physiological buffer or water;

(d) a light source for exciting the at least one compound or group of compounds according to (a); and

(e) a detection system for detecting the emitted electromagnetic radiation according to (b).

In another preferred embodiment of the method of the invention, at least one test substance is added prior to step (a) and/or prior to step (b) and/or prior to step (c) and/or prior to step (d), in order to observe whether the test substance influences the binding event. In this way, it is possible to determine substances which act on the interaction between CRP or C1q and a binder, in particular on the interaction between CRP and C1q, in a modulating, inhibiting or activating manner. This preferably involves screening a library of test substances in the HTS method in order to determine a substance having the desired properties.

The present invention therefore likewise relates to an HTS-capable assay system for determining active substances which act on the interaction between CRP or C1q and a component binding to CRP or C1q in a modulating manner, comprising the following components:

(a) at least one donor component or group of components containing at least one donor component, the donor component comprising at least one compound or group of compounds capable of emitting a signal after excitation by a light source (donor group) and the donor component being capable of binding directly or via one or more components from the group of components to CRP or C1q; and

(b) at least one acceptor component or group of components containing at least one at least one compound or group of compounds capable of receiving and emitting in the form of electromagnetic radiation the signal emitted by the compound or group of compounds according to (a) and the acceptor component being capable of binding directly or via one or more components of the group of components to CRP or C1q;

(c) at least one test compound.

The HTS-capable assay system here comprises preferably the following additional components:

(d) a light source for exciting the at least one compound or group of compounds according to (a);

(e) a detection system for detecting the emitted electromagnetic radiation according to (b); and

(f) a CRP- or C1q-containing solution.

The HTS-capable assay systems of the invention are used here preferably for carrying out the above-described methods of the invention. Particular embodiments of the parts and components of the assay systems therefore correspond to the abovementioned particular embodiments employed in the methods of the invention.

### EXAMPLES

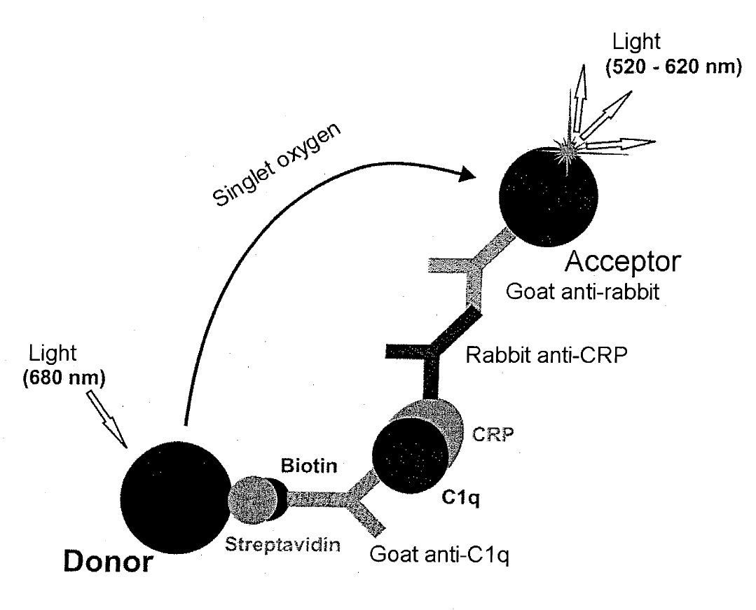

Binding assay for determining binding of CRP to C1q FIG. 1 shows the method according to the embodiment described below. Here, the donor component used was an anti-C1q antibody, the donor group being located on particles bound to the anti-C1q antibody. In the present case, the donor component can bind via C1q to the CRP. The acceptor component in the present case is an anti-rabbit secondary antibody, the acceptor group being located on particles bound to the anti-rabbit secondary antibody. In the present case, the acceptor component can bind via an anti-CRP antibody from rabbit to the CRP.

The experiment was carried out using the Alphascreen Detection Kit from Packard Bioscience which comprises donor and acceptor beads, the donor beads comprising compounds which, after excitation at a defined wavelength, can convert triplet oxygen to singlet oxygen, and the acceptor beads comprising compounds excitable by singlet oxygen, and also compounds capable of receiving in the form of an emissionless energy transfer and then emitting in the form of fluorescence radiation energy from the excited compounds. The compounds mentioned are embedded here in each case in a hydrogel matrix. The beads used had already been precoated by the manufacturer, the donor beads with streptavidin and the acceptor beads with anti-rabbit antibody.

TABLE 1
Overview of the materials used

| | MOLECULAR WEIGHT OR | SUPPLIER, CATALOG |
|---|---|---|
| REAGENTS/PLATES | CONCENTRATION | NUMBER |
| Microplate, 384K, PS, white | | Greiner 784075 |
| C-reactive protein, human, | 1 mg/ml, 115 kd (5mer) | Calbiochem 236608 |
| recombinant from E. coli | | |
| C1q, human | 1 mg/ml, 410 kd | Calbiochem 204876 |
| Anti-CRP, rabbit | 3.57 mg/ml, 165 kd | Caibiochem 235752 |
| AlphaScreen IgG detection | | Packard BioScience |
| kit (Protein A) | | 6760617 |
| AlphaScreen Rabbit IgG | | Packard BioScience |
| detection kit | | 6760607 |
| Anti-C1q, goat | Polyclonal serum | Calbiochem 234390 |
| Anti-streptavidin | Polyclonal serum | Sigma S6390 |
| EZ-Link Sulfo-NHS-LC- | | Pierce 21430 |
| biotinylation kit | | |
| Sodium chloride | 58.44 g/mol | Merck 101540 |
| Calcium chloride (CaCl₂ × 2 | 147.02 g/mol | Merck 102382 |
| H₂O) | | |
| Tris(hydroxymethyl)- | 157.6 g/mol | Merck 108219 |
| aminomethanehydrochloride | | |
| (Tris) | | |
| Bovine serum albumin (BSA) | | Sigma P7888 |
| Phosphoryl choline chloride | 329.7 g/mol | Sigma P0378 |
| Phosphate-buffered saline | | Gibco BRL 14200-067 |
| (Mg²⁺ + and Ca²⁺ -free) (PBS) | | |
| Slide-A-Lyzer mini dialysis | | Pierce P .69570 |
| units 10,000 MWCO | | |
| Dimethyl sulfoxide (DMSO) | | Merck KGaA, 1.02931 |
| Ethanol | | Riedel de Haen, 32250 |
| Pluronic F-68 | 10% strength solution | Sigma, P5556 |
| Hydrochloric acid | 1 M | Merck, 109057 |
| Sodium hydroxide solution | 1 M | Merck, 109137 |
| Milli-Q-H20 | | |

### Preparation of the Reaction Buffer

The following reaction buffer was used in the Alpha-Screen-System: 20 mM Tris-HCl, pH 7.2, 150 mM NaCl, 5 mM CaCl₂, 1 mM phosphoryl choline, 0.1% BSA. A stock solution of Tris-NaCl buffer was prepared by dissolving 3.15 g of Tris-HCl and 8.77 g of NaCl in H₂O, adjusting the pH to 7.2 by means of 1 M NaOH and then adding H2O to 1 l. The buffer is stored at room temperature. The reaction buffer was prepared by adding 36.7 mg of CaCl₂, 12.9 mg of phosphoryl choline and 50 mg of BSA to 50 ml of Tris-NaCl buffer.

Biotinylation of the Anti-C1q Antibody

In order to enable the anti-C1q antibody to bind to the streptavidin-coated donor beads, the former were biotinylated first. To this end, 100μl of anti-C1q polyclonal serum were dialyzed twice against 200 ml of PBS at room temperature for 1 hour. After adding 20 μl of 1.5 mM sulfo-NHS solution (in Milli-Q water), the solution was left standing at room temperature for 30 minutes and then dialyzed again twice against PBS, in order to remove excess biotinylation reagent.

### Assay Procedure

The assay was carried out by initially introducing 2 μl of donor bead/anti-CRP solution and then adding 2 μl of C1q, 2 μl of CRP and 2 μl of acceptor bead/anti-CRP. This resulted in the following final concentrations: CRP: 1 nM; C1q: 10 nM; anti-CRP: 7.3 nM; anti-C1q: 1:1500; donor beads: 20 μg/ml; acceptor beads: 40 μg/ml. In the negative control, reaction buffer replaced the CRP solution, the C1q solution or both of these solutions. After incubating at room temperature for 2 hours, the data were read out by means of an AlphaQuest reader.

### Results

The measured intensities of the emitted radiation at a CRP concentration of 1 nM and a C1q concentration of 10 nM were as follows (photomultiplier counts are listed):

In the absence of CRP and absence of C1q: 952

In the presence of CRP and absence of C1q: 1255

In the absence of CRP and presence of C1q: 1114

In the presence of CRP and presence of C1q: 80376

As can be seen, the negative controls did not provide a positive result, while a strong signal is visible when the C1q- and CRP-containing solutions are combined.

What is claimed is:

1. A method for detecting a binding event between at least two components and C-reactive protein, the method comprising the steps of
introducing a solution containing C-reactive protein;
adding to the solution at least one donor component comprising at least one compound that binds C-reactive protein and emits a signal after a light source excites the compound;
adding to the solution at least one acceptor component comprising at least one compound that binds C-reactive protein and that receives the signal and emits it in the form of electromagnetic radiation.
exciting the donor component with a light source;
detecting the electromagnetic radiation emitted by the acceptor component.

2. The method of claim 1, wherein the step of adding the at least one donor component further comprises adding a group of donor components.

3. The method of claim 1, wherein the at least one donor component further comprises a group of compounds that emit a signal after a light source excites them.

4. The method of claim 1, wherein the at least one donor component binds directly via one or more components to C-reactive protein.

5. The method of claim 1, wherein the at least one donor component binds indirectly via one or more components to C-reactive protein.

6. The method claim 1, wherein the step of adding the at least one acceptor component further comprises adding a group of acceptor components.

7. The method of claim 1, wherein the at least one acceptor component further comprises a group of compounds.

8. The method of claim 1, wherein the at least one acceptor component binds directly via one or more components to C-reactive protein.

9. The method of claim 1, wherein the at least one acceptor component binds indirectly via one or more components to C-reactive protein.

10. The method of claim 1, wherein the electromagnetic radiation is fluorescence radiation.

11. The method of claim 1, wherein the light source is selected from the group consisting of a laser and a lamp.

12. The method of claim 1, further comprising detecting with a photomultiplier the electromagnetic radiation emitted by the at least one acceptor component.

13. The method of claim 1, wherein the at least one donor component and at least one acceptor component bind, in spatial succession, to the C-reactive protein or to each other.

14. The method of claim 1, wherein the at least one donor component and at least one acceptor component comprise polypeptides.

15. The method of claim 14, wherein at least one of the components is an antibody.

16. The method of claim 15, wherein the antibody is selected from the group consisting of a monoclonal or polyclonal antibody.

17. The method of claim 1, wherein at least one of the components is a natural binding partner of the C-reactive protein.

18. The method of claim 17, wherein the natural binding partner is C1q.

19. The method of claim 18, wherein the at least one donor component or at least one acceptor component comprises C1q and an anti-C1q antibody.

20. The method of claim 1, wherein the signal is transferred from the at least one donor component to the at least one acceptor component by emissionless energy transfer.

21. The method of claim 20, wherein the emissionless energy transfer is a fluorescence resonance energy transfer.

22. The method of claim 1, wherein the signal is transferred from the at least one donor component to the at least one acceptor component via singlet oxygen.

23. The method of claim 1, wherein the at least one donor component comprises a compound which is able to convert triplet oxygen to singlet oxygen after excitation by a laser and wherein the at least one acceptor group comprises at least one first compound excitable by singlet oxygen and at least one second compound capable of absorbing in an emissionless manner and emitting in the form of fluorescence radiation the energy absorbed by the first compound.

24. The method of claim 23, wherein the donor group and the acceptor group are localized on particles.

25. The method of claimed in claim 24, wherein the particles have an average diameter of approximately 200 nm.

26. The method of claim 1, further comprising the step of determining the concentration of C-reactive protein in the solution.

27. The method of claim 26, wherein the solution is selected from the group consisting of blood serum, blood plasma, blood serum diluted by a physiological buffer, blood serum diluted by water, blood plasma diluted by a physiological buffer, and blood plasma diluted by water.

28. The method of claim 1, further comprising the steps of
adding at least one test substance; and
observing whether the test substance influences the binding event between C-reactive protein and the at least two components.

29. A method for detecting a binding event between at least two components and C1q, the method comprising the steps of
introducing a solution containing C1q;
adding to the solution at least one donor component comprising at least one compound that binds C1q and emits a signal after a light source excites the compound;
adding to the solution at least one acceptor component comprising at least one compound that binds C1q and that receives the signal and emits it in the form of electromagnetic radiation.
exciting the donor component with a light source;
detecting the electromagnetic radiation emitted by the acceptor component.

30. The method of claim 29, wherein the step of adding the at least one donor component further comprises adding a group of donor components.

31. The method of claim 29, wherein the at least one donor component further comprises a group of compounds that emit a signal after a light source excites them.

32. The method of claim 29, wherein the at least one donor component binds directly via one or more components to C1q.

33. The method of claim 29, wherein the at least one donor component binds indirectly via one or more components to C1q.

34. The method claim 29, wherein the step of adding the at least one acceptor component further comprises adding a group of acceptor components.

35. The method of claim 29, wherein the at least one acceptor component further comprises a group of compounds.

36. The method of claim 29, wherein the at least one acceptor component binds directly via one or more components to C1q.

37. The method of claim 29, wherein the at least one acceptor component binds indirectly via one or more components to C1q.

38. The method of claim 29, wherein the electromagnetic radiation is fluorescence radiation.

39. The method of claim 29, wherein the light source is selected from the group consisting of a laser and a lamp.

40. The method of claim 29, further comprising detecting with a photomultiplier the electromagnetic radiation emitted by the at least one acceptor component.

41. The method of claim 29, wherein the at least one donor component and at least one acceptor component bind, in spatial succession, to the C1q or to each other.

42. The method of claim 29, wherein the at least one donor component and at least one acceptor component comprise polypeptides.

43. The method of claim 42, wherein at least one of the components is an antibody.

44. The method of claim 43, wherein the antibody is selected from the group consisting of a monoclonal or polyclonal antibody.

45. The method of claim 29, wherein at least one of the components is a natural binding partner of the C1q.

46. The method of claim 45, wherein the natural binding partner is C-reactive protein.

47. The method of claim 46, wherein the at least one donor component or at least one acceptor component comprises C-reactive protein and an anti-C-reactive protein antibody.

48. The method of claim 29, wherein the signal is transferred from the at least one donor component to the at least one acceptor component by emissionless energy transfer.

49. The method of claim 48, wherein the emissionless energy transfer is a fluorescence resonance energy transfer.

50. The method of claim 29, wherein the signal is transferred from the at least one donor component to the at least one acceptor component via singlet oxygen.

51. The method of claim 29, wherein the at least one donor component comprises a compound which is able to convert triplet oxygen to singlet oxygen after excitation by a laser and wherein the at least one acceptor group comprises at least one first compound excitable by singlet oxygen and at least one second compound capable of absorbing in an emissionless manner and emitting in the form of fluorescence radiation the energy absorbed by the first compound.

52. The method of claim 51, wherein the donor group or the acceptor group are localized on particles.

53. The method of claim 52, wherein the particles have an average diameter of approximately 200 nm.

54. The method of claim 29, further comprising the step of determining the concentration of C-reactive protein in the solution.

55. The method of claim 54, wherein the solution is selected from the group consisting of blood serum, blood plasma, blood serum diluted by a physiological buffer, blood serum diluted by water, blood plasma diluted by a physiological buffer, and blood plasma diluted by water.

56. The method of claim 29, further comprising the steps of
adding at least one test substance; and
observing whether the test substance influences the binding event between C-reactive protein and the at least two components.

57. An HTS-capable, diagnostic assay system for determining a binding event between C-reactive protein and a component binding to C-reactive protein, comprising:
at least one donor component comprising at least one compound that binds C-reactive protein and emits a signal after a light source excites the compound; and
at least one acceptor component comprising at least one compound that binds C-reactive protein and that receives the signal and emits it in the form of electromagnetic radiation.

58. The assay system of claim 57, further comprising a group of donor components.

59. The assay system of claim 57, wherein the at least one donor component further comprises a group of compounds that emit a signal after a light source excites them.

60. The assay system of claim 57, wherein the at least one donor component binds directly via one or more components to C-reactive protein.

61. The assay system of claim 57, wherein the at least one donor component binds indirectly via one or more components to C-reactive protein.

62. The assay system of claim 57, wherein the at least one acceptor component further comprises a group of acceptor components.

63. The assay system of claim 57, wherein the at least one acceptor component further comprises a group of compounds.

64. The assay system of claim 57, wherein the at least one acceptor component binds directly via one or more components to C-reactive protein.

65. The assay system of claim 57, wherein the at least one acceptor component binds indirectly via one or more components to C-reactive protein.

66. The assay system of claim 57, further comprising:
a solution selected from the group consisting of blood serum, blood plasma, blood serum diluted by a physiological buffer, blood serum diluted by water, blood plasma diluted by a physiological buffer, and blood plasma diluted by water;
a light source for exciting the at least one donor component; and
a detection system for detecting the electromagnetic radiation emitted by the at least one acceptor component.

67. An HTS-capable, diagnostic assay system for determining a binding event between C1q and a component binding to C1q, comprising:
at least one donor component comprising at least one compound that binds C1q and emits a signal after a light source excites the compound; and
at least one acceptor component comprising at least one compound that binds C1q and that receives the signal and emits it in the form of electromagnetic radiation.

68. The assay system of claim 67, further comprising a group of donor components.

69. The assay system of claim 67, wherein the at least one donor component further comprises a group of compounds that emit a signal after a light source excites them.

70. The assay system of claim 67, wherein the at least one donor component binds directly via one or more components to C1q.

71. The assay system of claim 67, wherein the at least one donor component binds indirectly via one or more components to C1q.

72. The assay system of claim 67, wherein the at least one acceptor component further comprises a group of acceptor components.

73. The assay system of claim 67, wherein the at least one acceptor component further comprises a group of compounds.

74. The assay system of claim 67, wherein the at least one acceptor component binds directly via one or more components to C1q.

75. The assay system of claim 67, wherein the at least one acceptor component binds indirectly via one or more components to C1q.

76. The assay system of claim 67, further comprising:
a solution selected from the group consisting of blood serum, blood plasma, blood serum diluted by a physiological buffer, blood serum diluted by water, blood plasma diluted by a physiological buffer, and blood plasma diluted by water;
a light source for exciting the at least one donor component; and a detection system for detecting the electromagnetic radiation emitted by the at least one acceptor component.
